(19) United States
(12) Patent Application Publication
(10) Pub. No.: US 2026/0284027 A1
(43) Pub. Date: Sep. 24, 2026

(54) USES OF SMALL-MOLECULE COMPOUND Cbl-b-IN-3 IN THE PREPARATION OF PRODUCTS FOR TREATING HEPATIC FIBROSIS

(71) Applicant: PEKING UNIVERSITY, Beijing (CN)

(72) Inventors: Xunde XIAN, Beijing (CN); Kaikai LU, Beijing (CN); Ling ZHANG, Beijing (CN); Yitong XU, Beijing (CN); Jingxuan CHEN, Beijing (CN); Guolin MIAO, Beijing (CN); Wei HUANG, Beijing (CN); Yuhui WANG, Beijing (CN)

(73) Assignee: PEKING UNIVERSITY, Beijing (CN)

(21) Appl. No.: 19/234,257

(22) Filed: Jun. 10, 2025

Related U.S. Application Data
(63) Continuation of application No. PCT/CN2025/087690, filed on Apr. 8, 2025.

(30) Foreign Application Priority Data
Mar. 20, 2025 (CN) 202510337248.5

(51) Int. Cl. A61K 31/454; A61P 1/16

(52) U.S. Cl. CPC A61K 31/454; A61P 1/16

(57) ABSTRACT

Provide is a method for treating hepatic fibrosis. The method includes administering a therapeutically effective amount of a small-molecule compound Cbl-b-IN-3 to a subject suffering from hepatic fibrosis. Cbl-b-IN-3 demonstrates a potent anti-hepatic fibrosis effect, showing significant therapeutic efficacy across multiple murine models of hepatic fibrosis, indicating broad applicability. Cbl-b-IN-3 upregulates expression of SMAD7—a key inhibitory factor in the TGF-β signaling pathway—and reduces activation of HSCs, thereby effectively alleviating hepatic fibrosis. This mechanism of action is distinct from existing anti-fibrotic drugs, representing a novel therapeutic strategy for hepatic fibrosis. Notably, within the effective dose range of 3-8 mg/kg/day, based on mouse body weight, Cbl-b-IN-3 displayed no significant toxic side effects, demonstrating a favorable safety profile. These findings collectively establish Cbl-b-IN-3 as a safe and effective anti-fibrotic agent capable of addressing the current limitations of suboptimal efficacy and pronounced adverse effects associated with available therapies.

### CROSS-REFERENCE

This application is a Continuation of International Application No. PCT/CN2025/087690, filed on Apr. 8, 2025, which claims priority to Chinese Patent Application No. 202510337248.5, filed on Mar. 20, 2025, the entire contents of which are incorporated herein by reference.

### SEQUENCE LISTING

The instant application contains a Sequence Listing which is submitted electronically in XML format and is hereby incorporated by reference in its entirety. The XML copy, created on May 6, 2025, is named “2025-5-6-Sequence Listing-65628-H040US00,” and is 6,421 bytes in size.

### TECHNICAL FIELD

The present disclosure relates to the field of biomedical technology, and in particular, to a use of a small-molecule compound Cbl-b-IN-3 (also referred to as NX-1607 by its development code) in the preparation of a product for treating hepatic fibrosis.

### BACKGROUND

Currently, hepatic fibrosis is a widespread pathological process caused by chronic liver injury, which may eventually progress to cirrhosis or even liver cancer. Although studies have shown that a TGF-β signaling pathway promotes the development of hepatic fibrosis, there are still no effective anti-fibrotic drugs targeting the TGF-β signaling pathway available in clinical practice.

Existing therapeutic strategies for hepatic fibrosis mainly include: (1) anti-inflammatory therapy, such as glucocorticoids, which may cause adverse effects such as immunosuppression when used long-term; (2) anti-oxidative stress therapy, such as S-adenosylmethionine, which has limited efficacy; and (3) Anti-fibrotic drugs, including some experimental TGF-β pathway inhibitors, most of which have not yet entered clinical application, and some of which exhibit poor pharmacokinetic properties or significant side effects.

Therefore, there is an urgent need for a safe and effective anti-hepatic fibrosis drug to address the limitations of the existing therapeutic strategies.

### SUMMARY

One or more embodiments of the present disclosure provide a method for treating hepatic fibrosis. The method includes administering a therapeutically effective amount of a small-molecule compound Cbl-b-IN-3 to a subject suffering from hepatic fibrosis.

In some embodiments, the small-molecule compound Cbl-b-IN-3 is part of a drug.

In some embodiments, a unit dose of the small-molecule compound Cbl-b-IN-3 is in a range from 3 mg/kg to 8 mg/kg mouse body weight.

In some embodiments, the unit dose of the small-molecule compound Cbl-b-IN-3 is 5 mg/kg mouse body weight.

In some embodiments, the drug further includes an excipient; and the excipient includes a solution of sodium carboxymethyl cellulose at a concentration of 5 g/L.

One or more embodiments of the present disclosure provide a drug for treating hepatic fibrosis. An active ingredient of the drug includes a small-molecule compound Cbl-b-IN-3, and a unit dose of the small-molecule compound Cbl-b-IN-3 is in a range from 3 mg/kg to 8 mg/kg mouse body weight.

In some embodiments, the unit dose of the small-molecule compound Cbl-b-IN-3 is 5 mg/kg mouse body weight.

In some embodiments, the drug further includes an excipient. The excipient includes a solution of sodium carboxymethyl cellulose at a concentration of 5 g/L.

### BRIEF DESCRIPTION OF THE DRAWINGS

The present disclosure will be further illustrated by way of exemplary embodiments, which will be described in detail by means of the accompanying drawings. These embodiments are not limiting, wherein:

FIG. 1 is a schematic diagram illustrating results of Masson staining in a methionine-choline deficient (MCD) diet-induced model after treatment with NX-1607 according to some embodiments of the present disclosure;

FIG. 2 is a schematic diagram illustrating results of Sirius Red staining in an MCD diet-induced model after treatment with NX-1607 according to some embodiments of the present disclosure;

FIG. 3 is a schematic diagram illustrating detection results of serum liver injury markers, alanine aminotransferase (ALT) and aspartate aminotransferase (AST), in an MCD diet-induced model after treatment with NX-1607 according to some embodiments of the present disclosure;

FIG. 4 is a schematic diagram illustrating RT-qPCR detection results in an MCD diet-induced model after treatment with NX-1607 according to some embodiments of the present disclosure;

FIG. 5 is a schematic diagram illustrating results of Masson staining in a carbon tetrachloride (CCl₄)-induced mode after treatment with NX-1607 according to some embodiments of the present disclosure;

FIG. 6 is a schematic diagram illustrating results of Sirius Red staining in a CCl₄-induced model after treatment with NX-1607 according to some embodiments of the present disclosure;

FIG. 7 is a schematic diagram illustrating detection results of serum liver injury markers, ALT and AST, in a CCl₄-induced model after treatment with NX-1607 according to some embodiments of the present disclosure;

FIG. 8 is a schematic diagram illustrating RT-qPCR detection results in a CCl₄-induced model after treatment with NX-1607 according to some embodiments of the present disclosure; and

FIG. 9 is a schematic diagram illustrating Western Blot detection results after stimulation of a human hepatic stellate cell line LX2 with NX-1607 according to some embodiments of the present disclosure.

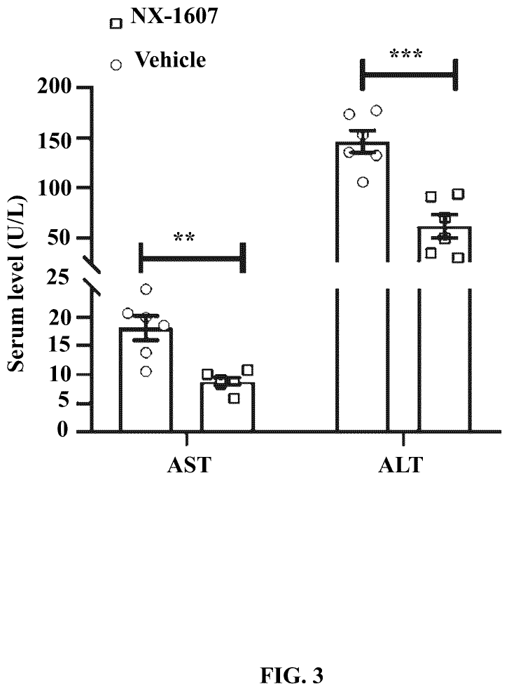

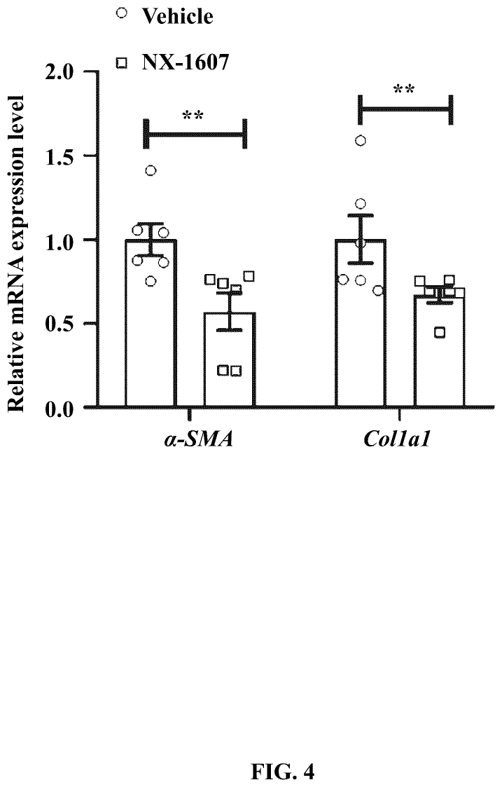

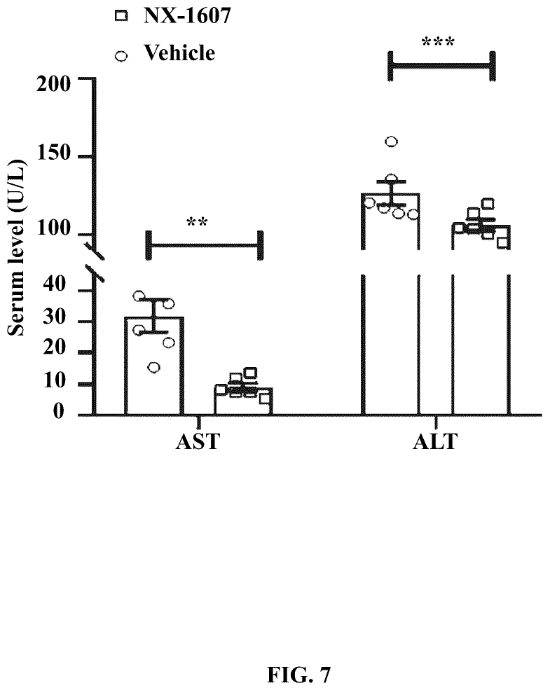

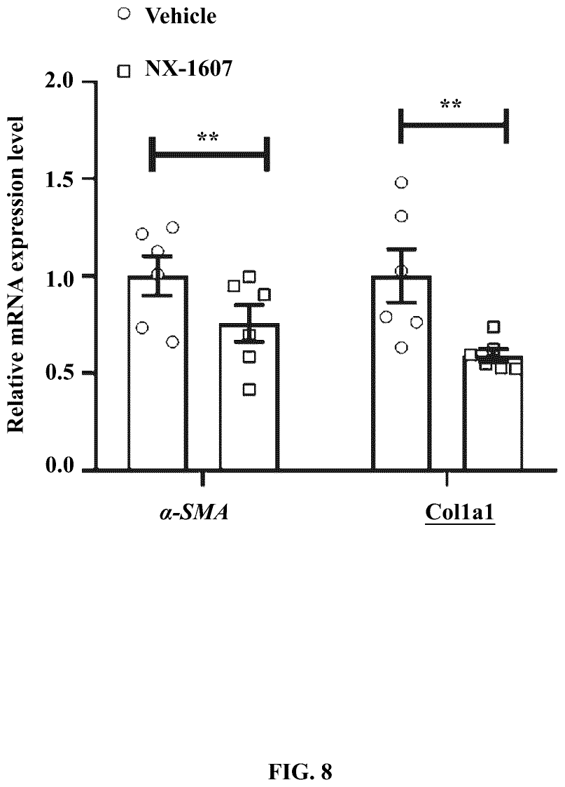

NX1607 in each of FIG. 3, FIG. 4, FIG. 7, and FIG. 8 refers to a NX-1607 treatment group.

### DETAILED DESCRIPTION

The present disclosure provides a use of a small-molecule compound Cbl-b-IN-3 in the preparation of a product for treating hepatic fibrosis. The small-molecule compound Cbl-b-IN-3 used in the present disclosure is a commercially available small-molecule compound currently in the clinical trial phase. At an effective dose range of 3-8 mg/kg/day based on mouse body weight, no significant toxic side effects have been observed, indicating good safety. In some embodiments, the Cbl-b-IN-3 used in the present disclosure is purchased from Selleck Chemicals, with catalog number E1957.

In some embodiments, the product described above may be a drug.

The present disclosure finds that Cbl-b-IN-3 demonstrates a potent anti-hepatic fibrosis effect in a plurality of mouse models of hepatic fibrosis, including an MCD diet-induced model and a CCl₄-induced model. The MCD diet-induced model is a model of hepatic fibrosis associated with non-alcoholic steatohepatitis (NASH). The CCl₄ is a classical chemical inducer of liver injury, which may be used to simulate drug-induced, alcohol-related, and viral hepatitis-mediated hepatic fibrosis. These findings suggest that Cbl-b-IN-3 has broad applicability and holds promise as a novel candidate for anti-fibrotic therapy.

The present disclosure also finds that Cbl-b-IN-3 may upregulate the expression of SMAD7, a key inhibitory factor of a TGF-β signaling pathway, thereby reducing the activation of hepatic stellate cells (HSCs) and effectively alleviating hepatic fibrosis. This mechanism differs from the mechanisms of currently available anti-fibrotic drugs and provides new technical support for treating hepatic fibrosis.

Based on the above advantages, the present disclosure provides a drug for treating hepatic fibrosis. An active ingredient of the drug includes the small-molecule compound Cbl-b-IN-3. A unit dose of the small-molecule compound Cbl-b-IN-3 is in a range from 3 mg/kg to 8 mg/kg mouse body weight. As used herein, the “unit dose” refers to an effective unit dose of the drug to exert an anti-hepatic fibrosis effect. Equivalent doses for human application may be calculated based on common knowledge in the art.

In some embodiments, the unit dose of the drug is 5 mg/kg mouse body weight.

In some embodiments, the drug further includes an excipient. The excipient includes a solution of sodium carboxymethyl cellulose at a concentration of 5 g/L.

To address the aforementioned issues, the present disclosure provides a use of the small-molecule compound Cbl-b-IN-3 in the preparation of a product for treating hepatic fibrosis. The present disclosure finds that Cbl-b-IN-3 may upregulate the expression of SMAD7, a key inhibitory factor of the TGF-β signaling pathway, and reduce the activation of the HSCs, thereby effectively alleviating hepatic fibrosis. Cbl-b-IN-3 is a safe and effective anti-hepatic fibrosis drug that can improve the current limitations of limited efficacy and significant side effects.

One or more embodiments of the present disclosure provide a use of the small-molecule compound Cbl-b-IN-3 in the preparation of a product for treating hepatic fibrosis.

In some embodiments, the product includes a drug.

One or more embodiments of the present disclosure provide a method for treating hepatic fibrosis. The method includes administering a therapeutically effective amount of the small-molecule compound Cbl-b-IN-3 to a subject suffering from hepatic fibrosis. The subject may be an animal (e.g., a mouse) or a human (which may be referred to as a patient).

In some embodiments, the small-molecule compound Cbl-b-IN-3 is part of a drug. In some embodiments, the drug is a composition.

In some embodiments, the unit dose of the small-molecule compound Cbl-b-IN-3 may be in a range from 3 mg/kg to 8 mg/kg mouse body weight. In some embodiments, the unit dose of the small-molecule compound Cbl-b-IN-3 may be 3 mg/kg, 4 mg/kg, 5 mg/kg, 6 mg/kg, 7 mg/kg, or 8 mg/kg mouse body weight.

In some embodiments, the unit dose of the small-molecule compound Cbl-b-IN-3 may be in a range from 0.24 mg/kg to 0.64 mg/kg patient body weight. In some embodiments, the unit dose of the small-molecule compound Cbl-b-IN-3 may be 0.24 mg/kg, 0.32 mg/kg, 0.40 mg/kg, 0.48 mg/kg, 0.56 mg/kg, or 0.64 mg/kg patient body weight.

In some embodiments, the unit dose of the small-molecule compound Cbl-b-IN-3 may be 5 mg/kg mouse body weight.

In some embodiments, the unit dose of the small-molecule compound Cbl-b-IN-3 may be 0.40 mg/kg patient body weight.

In some embodiments, the drug further includes an excipient, and the excipient includes a solution of sodium carboxymethyl cellulose at a concentration of 5 g/L.

One or more embodiments of the present disclosure provide a drug for treating hepatic fibrosis. An active ingredient of the drug includes the small-molecule compound Cbl-b-IN-3, and a unit dose of the drug is in a range from 3 mg/kg to 8 mg/kg mouse body weight.

In some embodiments, the unit dose of the small-molecule compound Cbl-b-IN-3 may be in a range from 0.24 mg/kg to 0.64 mg/kg patient body weight.

In some embodiments, the unit dose of the drug may be 3 mg/kg, 4 mg/kg, 5 mg/kg, 6 mg/kg, 7 mg/kg, or 8 mg/kg mouse body weight.

In some embodiments, the unit dose of the drug may be 0.24 mg/kg, 0.32 mg/kg, 0.40 mg/kg, 0.48 mg/kg, 0.56 mg/kg, or 0.64 mg/kg patient body weight.

In some embodiments, the unit dose of the drug is 5 mg/kg mouse body weight.

In some embodiments, the unit dose of the drug is 0.40 mg/kg patient body weight.

In some embodiments, the drug further includes an excipient, and the excipient includes a solution of sodium carboxymethyl cellulose at a concentration of 5 g/L.

In some embodiments of the present disclosure, Cbl-b-IN-3 is found to have highly efficient anti-hepatic fibrosis effects. In a plurality of mouse models of hepatic fibrosis (e.g., the MCD diet-induced model and the CCl₄-induced model), Cbl-b-IN-3 consistently showed significant anti-fibrotic activity by suppressing αSMA gene expression and Colla1 gene expression, and reduced serum levels of alanine aminotransferase (ALT) and aspartate aminotransferase (AST). These findings indicate broad applicability and potential of Cbl-b-IN-3 as a novel anti-fibrotic drug candidate. The present disclosure also finds that Cbl-b-IN-3 upregulates the expression of SMAD7, a key inhibitory factor of the TGF-β signaling pathway, and reduces the activation of the HSCs, thereby effectively alleviating hepatic fibrosis. The mechanism differs from mechanisms of existing anti-hepatic fibrosis drugs, providing new technical support for treating hepatic fibrosis. In addition, Cbl-b-IN-3 is a commercially available drug entering the clinical stage. Within an effective dose range (3 mg/kg/d to 8 mg/kg/d mouse body weight), no significant toxic side effects were observed, demonstrating favorable safety profiles. In summary, the present disclosure finds that Cbl-b-IN-3 is a safe and effective anti-hepatic fibrosis drug that can address current limitations of limited efficacy and significant adverse effects associated with existing therapies.

To further illustrate the present disclosure, the use of the small-molecule compound Cbl-b-IN-3 (also referred to as NX1607 or NX-1607) provided herein in the preparation of a product for treating hepatic fibrosis is described in detail below in conjunction with the accompanying drawings and examples. However, these descriptions should not be construed as limiting the scope of protection of the present disclosure.

### EXAMPLES

### Example 1

Adopting the approach established by Rao J, Wang H, Ni M, Wang Z, Wang Z, Wei S, Liu M, Wang P, Qiu J, Zhang L, Wu C, Shen H, Wang X, Cheng F, Lu L in “FSTL1 promotes hepatic fibrosis by reprogramming macrophage function through modulating the intracellular function of PKM2” (Gut. 2022 December; 71(12):2539-2550. doi: 10.1136/gutjnl-2021-325150. Epub 2022 Feb. 9. PMID: 35140065; PMCID: PMC9664121.), C57BL/6J mice were subjected to hepatic fibrosis modeling, generating two mouse hepatic fibrosis models: an MCD diet-induced model and a CCl₄-induced model).

### Example 2

NX-1607 (also denoted as NX1607, purchased from Selleck Chemicals) was dissolved in a solution of sodium carboxymethyl cellulose at a concentration of 5 g/L to obtain an NX-1607 solution.

The MCD diet-induced model mice constructed in Example 1 were divided into two groups, a control group (denoted as Vehicle) and an NX-1607 treatment group, with six mice in each group. Two groups of mice were treated as follows.

Mice in the NX-1607 treatment group received daily oral gavage of the NX-1607 solution at a dose of 5 mg/kg body weight (based on NX-1607 mass), and mice in the control group received daily oral gavage of an equal volume of vehicle (i.e., the solution of sodium carboxymethyl cellulose at a concentration of 5 g/L).

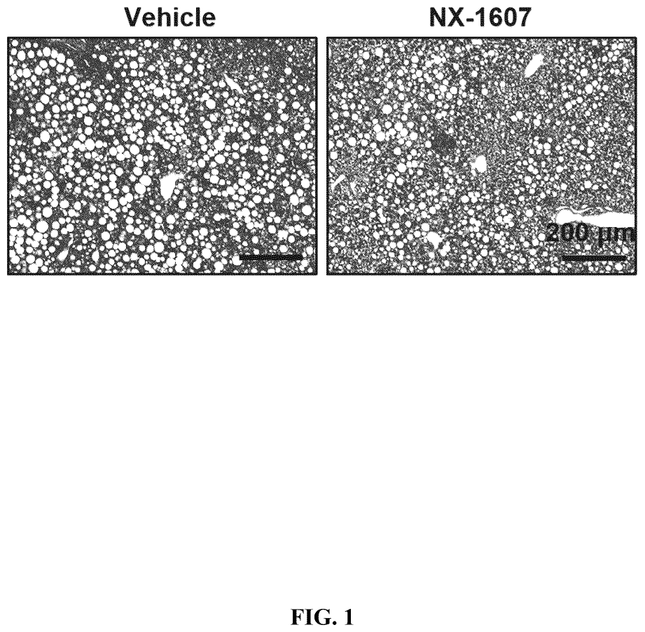

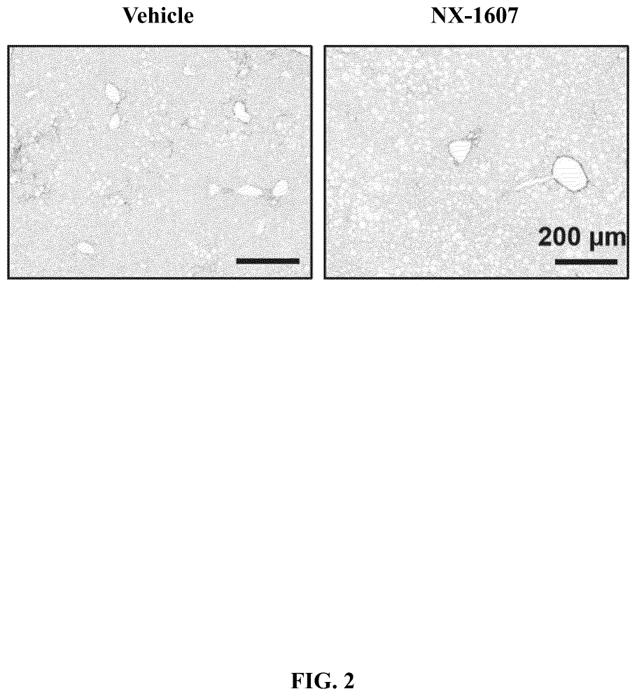

After six weeks of treatment, liver paraffin sections from the mice of the NX-1607 treatment group and the control group were subjected to Masson's trichrome staining and Sirius red staining. The results are shown in FIG. 1 and FIG. 2. The results show that a degree of hepatic fibrosis was reduced in the MCD diet-induced model mice after NX-1607 treatment.

After six weeks of treatment, serum levels of liver injury markers—alanine aminotransferase (ALT) and aspartate aminotransferase (AST)—were measured in both groups. The results are presented in FIG. 3, where ** indicates P<0.01 and *** indicates P<0.001. The results show that NX-1607 treatment significantly decreased the serum levels of the liver injury markers (ALT and AST) in MCD diet-induced model mice.

After six weeks of treatment, relative expression levels of hepatic fibrosis molecular markers (αSMA and Colla1 genes) were detected by RT-qPCR, using actin as an internal reference gene. Primer sequences used are listed in Table 1. The results are shown in FIG. 4, where ** indicates P<0.01.

TABLE 1
RT-qPCR primer sequences

| Primer Name | Primer Sequence (5′→3′) |
|---|---|
| mus-actin-F | GGCTGTATTCCCCTCCATCG |
| | (SEQ ID NO: 1) |
| mus-actin-R | CCAGTTGGTAACAATGCCATGT |
| | (SEQ ID NO: 2) |
| mus-αSMA-F | CCCAGACATCAGGGGAGTAATGGG |
| | (SEQ ID NO: 3) |
| mus-αSMA-R | TCTATCGGATACTTCAGCGTCA |
| | (SEQ ID NO: 4) |
| mus-Col1a1- | TGCTAACGTGGTTCGTGACCGT |
| F | (SEQ ID NO: 5) |
| mus-Col1a1- | ACATCTTGAGGTCGCGGCATGT |
| R | (SEQ ID NO: 6) |

The results show that the relative expression levels of the molecular markers, hepatic fibrosis αSMA gene and Colla1 gene, were reduced in the MCD diet-induced model mice after NX-1607 treatment.

### Example 3

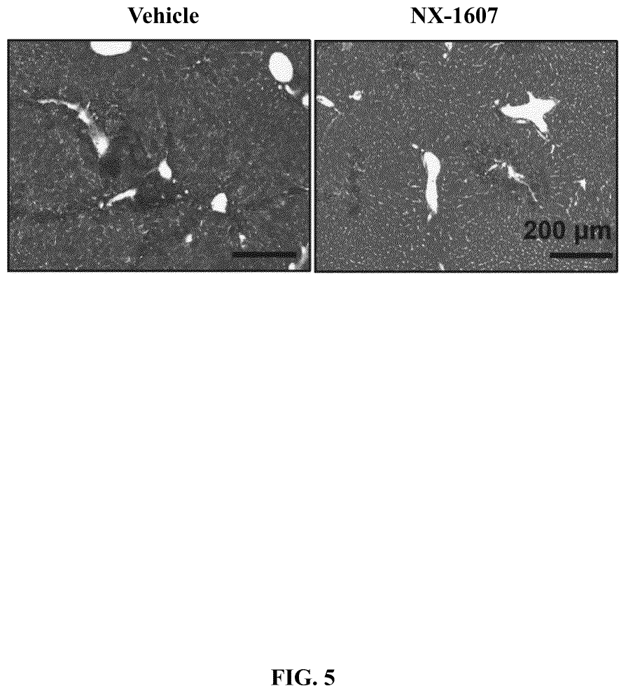

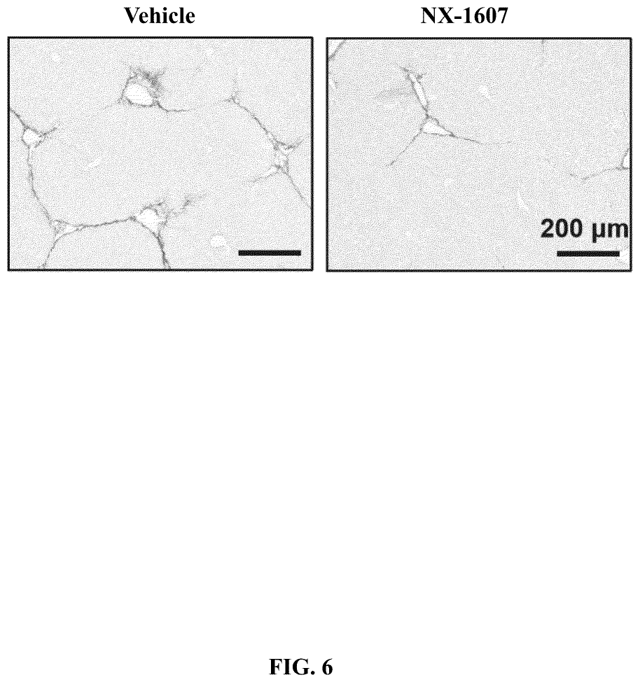

Following a similar method to Example 2, with the modification that the MCD diet-induced model mice from Example 1 were replaced with CCl₄-induced model mice from Example 1. The results are shown in FIGS. 5-8, where ** indicates p<0.01 and *** indicates P<0.001.

The results demonstrate that NX-1607 treatment reduced: the degree of hepatic fibrosis in CCl₄-induced model mice, the serum levels of liver injury markers (ALT and AST), and the relative expression levels of hepatic fibrosis molecular markers (the αSMA and Colla1 genes).

### Example 4

The human hepatic stellate cell line LX2 was stimulated separately with transforming growth factor-β (TGF-β) at a concentration of 10 ng/mL and with NX-1607 at a concentration of 100 nM (dissolved in a 5 g/L sodium carboxymethyl cellulose solution) for 24 hours. Total cellular protein was then extracted and subjected to Western blot analysis. The specific Western blot procedure is as follows:

### 1. Cell Lysis and Protein Extraction

Cells were washed two times with a pre-cooled phosphate buffer solution (PBS) to remove medium residues. An appropriate amount of Radio Immunoprecipitation Assay (RIPA) lysis buffer (containing a protease inhibitor and a phosphatase inhibitor) was added and placed on ice for thirty minutes, with vortexing every 10 min. Centrifugation was performed at 12,000×g at 4° C. for fifteen minutes, and the supernatant (i.e., total protein) was collected. Protein concentration was determined by using a bicinchoninic acid (BCA) protein quantification kit. 15 g of protein samples are added into 5× protein loading buffer (SDS-PAGE loading buffer) and denatured at 100° C. for five minutes.

### 2. SDS-PAGE Electrophoresis

A 10% SDS-PAGE gel was prepared and polymerized. Samples were loaded into wells of the gel, a protein molecular weight marker was added, and electrophoresis was performed at 80 V for thirty minutes and then 120 V for sixty minutes until the proteins were separated to the proper locations.

### 3. Protein Transfer

The gel-separated proteins were transferred to a 0.22 μm polyvinylidene difluoride (PVDF) membrane via wet transfer at 400 mA for sixty minutes).

### 4. Blocking and Antibody Incubation

The membrane was blocked with 5% skim milk at room temperature for two hours. Primary antibodies against SMAD7 and glyceraldehyde-3-phosphate dehydrogenase (GAPDH) (at a dilution ratio of 1:1000) were added and incubated at 4° C. overnight. The membrane was then washed three times with TBST (Tris-Borate-Sodium Tween-20 buffer), 10 minutes each time. A Horseradish Peroxidase (HRP)-conjugated secondary antibody (at a dilution ratio 1:5000) was added and incubated at room temperature for one hour, followed by three additional TBST washes, 10 minutes each.

### 5, Enhanced Chemiluminescence (ECL) Color Development and Signal Detection

An ECL luminescent substrate was added dropwise on the membrane and incubated for one minute in the dark. Protein bands were detected using a chemiluminescence imaging system or X-ray film. Target protein expression was normalized against GAPDH band intensity.

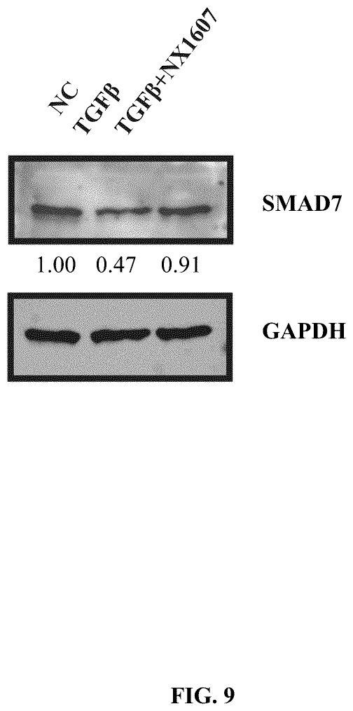

The results are shown in FIG. 9. Compared with TGF-β stimulation (0.47), NX-1607 promoted SMAD7 expression (0.91) in HSCs.

In summary, the present disclosure provides a safe and effective anti-hepatic fibrosis small-molecule drug, NX-1607. NX-1607 alleviates liver fibrosis by promoting the expression of SMAD7, a key inhibitory factor in the TGF-β signaling pathway, thereby reducing the activation of hepatic HSCs, and thus addressed the limitations of current therapeutic approaches.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

It should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This way of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameter set forth in the written description and attached claims are approximations that may vary depending upon the desired properties sought to be obtained by a particular embodiment. In some embodiments, the numerical parameter should be construed in light of the count of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for treating hepatic fibrosis, comprising administering a therapeutically effective amount of a small-molecule compound Cbl-b-IN-3 to a subject suffering from hepatic fibrosis.

2. The method of claim 1, wherein the small-molecule compound Cbl-b-IN-3 is part of a drug.

3. The method of claim 2, wherein a unit dose of the small-molecule compound Cbl-b-IN-3 is in a range from 3 mg/kg to 8 mg/kg mouse body weight.

4. The method of claim 2, wherein a unit dose of the small-molecule compound Cbl-b-IN-3 is 5 mg/kg mouse body weight.

5. The method of claim 2, wherein the drug further includes an excipient, and the excipient includes a solution of sodium carboxymethyl cellulose at a concentration of 5 g/L.

6. A drug for treating hepatic fibrosis, wherein an active ingredient of the drug includes a small-molecule compound Cbl-b-IN-3, and a unit dose of the small-molecule compound Cbl-b-IN-3 is in a range from 3 mg/kg to 8 mg/kg mouse body weight.

7. The drug of claim 6, wherein the unit dose of the small-molecule compound Cbl-b-IN-3 is 5 mg/kg mouse body weight.

8. The drug of claim 6, further comprising an excipient, wherein the excipient includes a solution of sodium carboxymethyl cellulose at a concentration of 5 g/L.


SEQUENCE LISTING (6 sequences)

SEQ ID NO: 1 - DNA, 20 bp, synthetic construct
  source 1..20: mol_type = other DNA
ggctgtattcccctccatcg

SEQ ID NO: 2 - DNA, 22 bp, synthetic construct
  source 1..22: mol_type = other DNA
ccagttggtaacaatgccatgt

SEQ ID NO: 3 - DNA, 24 bp, synthetic construct
  source 1..24: mol_type = other DNA
cccagacatcaggggagtaatggg

SEQ ID NO: 4 - DNA, 22 bp, synthetic construct
  source 1..22: mol_type = other DNA
tctatcggatacttcagcgtca

SEQ ID NO: 5 - DNA, 22 bp, synthetic construct
  source 1..22: mol_type = other DNA
tgctaacgtggttcgtgaccgt

SEQ ID NO: 6 - DNA, 22 bp, synthetic construct
  source 1..22: mol_type = other DNA
acatcttgaggtcgcggcatgt